(19) United States
(12) Patent Application Publication
(10) Pub. No.: US 2010/0023045 A1
(43) Pub. Date: Jan. 28, 2010

(54) Lancet, Lancet Supply Ribbon, and Puncturing Device for Generating a Puncturing Wound

(76) Inventors: Heinz Macho, Furth (DE); Josef Roeper, Neuhofen (DE)

Correspondence Address: BAKER & DANIELS LLP / ROCHE, 300 NORTH MERIDIAN STREET, SUITE 2700, INDIANAPOLIS, IN, 46204, US

(21) Appl. No.: 11/778,362

(22) Filed: Jul. 16, 2007

(30) Foreign Application Priority Data
Jul. 15, 2006 (EP) 06014792.3

(51) Int. Cl. A61B 17/32

(52) U.S. Cl. 606/182; 606/181

(57) ABSTRACT

The invention refers to a puncturing device for generating a puncturing wound by means of a replaceable lancet that comprises a coupling element for transmitting a torque, whereby the puncturing device comprises a lancet drive which, for a puncture, transmits a torque to an inserted lancet that is coupled to the lancet drive by means of its coupling element such that the inserted lancet performs a puncturing motion in the form of a rotational motion about a geometric axis. According to embodiments of the invention, the geometric axis extends through the coupling element. The invention also refers to a lancet supply ribbon comprising lancets for such device and lancets comprising a lancet body having a lancet tip, whereby the lancet body, for coupling to a lancet drive of a puncturing device, comprises a coupling element by means of which the lancet can be made to perform a rotational motion and has a test field for assaying a body fluid sample to which a body fluid sample can be supplied only after the lancet tip has been pulled out of a puncturing wound that has been generated.

### BACKGROUND OF THE INVENTION

1. Field of the Invention

This application is related to European Application Serial No. 06014792.3, filed Jul. 15, 2006, the disclosures of which are expressly incorporated by reference herein.

The invention is based on a puncturing device for generating a puncturing wound by means of a replaceable lancet that comprises a coupling element for transmitting a torque, whereby the puncturing device comprises a lancet drive which, for a puncture, transmits a torque to an inserted lancet that is coupled to the lancet drive by means of its coupling element.

2. Description of the Prior Art

A puncturing device and matching flat lancet are known from U.S. Pat. No. 5,951,582, the disclosure of which is expressly incorporated herein by reference. The coupling element of the known lancet is provided in the form of a slit that is engaged by a peg of the lancet drive. During a puncture, the peg slides within the slit and, in the process, transmits a torque to the lancet such that the tip of the lancet performs an arc-shaped motion.

Puncturing devices and corresponding lancets are needed, for example, by diabetics who need to check their blood sugar levels multiple times daily by generating a small puncturing wound in a body part, usually a finger, in order to obtain a body fluid sample that can be assayed using a measuring device to determine the glucose content. In order to render the determination of the glucose content as simple as possible for diabetics, lancets are known, for example from WO 2004/086970 A1, that are provided with a test field for assaying a body fluid sample obtained by means of a puncturing wound.

Being consumables, large numbers of lancets are needed, and lancets thus cause a substantial fraction of the costs associated with the treatment of diabetes. One very common option for the cost-efficient manufacture of lancets consists of punching a lancet body including a lancet tip from a metal ribbon such as described in WO 2004/086970 A1. Moreover, EP 1346686 A2 specifies the use of etching techniques for the manufacture of lancets from sheet metal. For example WO 2005/084530 proposes non-metallic materials, for example, silicon and ceramic materials, for the manufacture of the lancets as an alternative to sheet metal.

### SUMMARY OF THE INVENTION

It is therefore an object of the invention to devise a way of making it easier for diabetics to treat their disease.

In a puncturing device according to an embodiment of the invention, an inserted lancet performs, during a puncture, a rotational motion whose geometric rotation axis extends through the coupling element that is preferably provided in the form of an opening, e.g. a recess or a hole, having a contour that deviates from a circular shape. The lancet tip can be accelerated to particularly high speeds and generate a puncturing wound through a pecking motion that is associated with particularly little pain.

A lancet according to embodiments of the invention that comprises a lancet body having a lancet tip can be manufactured cost-efficiently, for example by cutting the lancet body including the lancet tip from a ribbon of metal using a laser.

In a metal body cut from a ribbon of metal by means of laser cutting, the cutting edges may show characteristic deposits made of metal and metal oxides that were melted and/or generated by the laser beam, respectively.

The use of laser cutting for small-lot production is a common procedure in various fields of engineering. With regard to small-lot numbers, laser cutting is usually more cost-efficient than punching. Accordingly, lancets can be manufactured more cheaply by means of laser cutting as compared to punching despite the extremely high production numbers.

In particular in the production of so-called integrated lancets that comprise a test field for assaying a body fluid sample obtained by means of a puncturing wound the use of a laser may generally afford savings of production costs. According to the state of the art, lancets of this type having a metallic lancet body and a test field for photometric or electrochemical determination of concentration necessitate a multitude of different fabrication technologies for the processing of, on the one hand, metallic components of the lancet, i.e. the lancet body and the lancet tip, and, on the other hand, non-metallic components, in particular the test field. These different technologies must be adapted to each other such that the investment costs for mass production increase strongly with the increase of the number of fabrication technologies employed in the process. In addition, each manufacturing step has only a limited plant efficiency such that a fabrication process combining multiple fabrication steps involving different technologies is associated with a risk of low overall plant efficiency.

In contrast to the fabrication technologies that are common according to the state of the art, such as etching or punching, a laser can be used not only for the processing of metallic lancet components, but also of non-metallic lancet components, such as, for example, a polymer fiber fabric containing test chemicals for a testing facility in the form of a test field that is integrated into the lancet. The use of a laser allows the lancet body, including the lancet tip, to be cut from sheet metal, a polymer fiber fabric to be cut to size and its edges to be melted together, and a lancet to be sealed in a film wrapping.

Test fields containing test chemicals that indicate the concentration of an analyte, for example glucose, by means of a change of color are very delicate. For this reason, lancets having integrated test fields must be stored in a dry place and protected from environmental influences until use. This can be realized by sealing the lancets individually in a film wrapping made of plastic material. A laser may be used for this manufacturing step.

Another feature of embodiments of the invention that can have independent significance for lancets manufactured by other means as well thus concerns a lancet supply ribbon comprising multiple lancets that are each sealed in chambers of a film wrapping, whereby the chambers of the film wrapping form a ribbon. A lancet supply ribbon of this type can be manufactured, for example, by arranging the lancets in a series on a plastic film, then covering them with a second film and sealing the two films, which are one over the other, to each other around the lancets such that a ribbon of airtight sealed chambers is formed.

As part of embodiments of the invention, it was noted that flat lancets, i.e. lancets cut from a ribbon of metal, can be removed from a film wrapping by means of a rotational motion. A lancet can also be used in a rotational motion to generate a puncturing wound in order to obtain a body fluid sample. In embodiments of the invention the arc-shaped rotational motion of the lancet tip that is performed as the puncturing motion sweeps over no more than 180°, in certain embodiments no more than 90°, and in other embodiments no more than 45°.

In embodiments of the invention, the coupling element is in the form of an opening in the lancet body. The opening can be coupled to a lancet drive of the puncturing device in a form-fitting fashion such that a torque can be transmitted from the lancet drive to the lancet. For the process of coupling, a drive element of the lancet drive, for example a shaft having a cross-section that matches the opening, is inserted into the opening of the lancet such that a rotational motion of the drive element is transmitted to the lancet. Generally, any contour of the opening that deviates from a circular shape is suitable for this purpose. However, in embodiments of the invention, the opening may have an angular, for example a star or square-shape contour.

Since the lancet body naturally is flat in shape, the inside surface of the opening can be very small and can be formed by an edge or line in the extreme case against which the drive element presses in order to transmit a torque.

In embodiments of the invention, lancets whose lancet body includes a lancet tip punched from a ribbon of metal or manufactured by other means may also include a coupling element by means of which the lancet can be made to perform a rotational motion, and of a corresponding puncturing device, in which the lancet drive transmits a torque to an inserted lancet for a puncture such that the lancet performs a puncturing motion in the form of a rotational motion.

Further details and advantages of the invention are illustrated in the following by means of exemplary embodiments and referring to the appended drawings. Equal or corresponding parts are identified by identical reference numbers. Features disclosed in the exemplary embodiments can be made the object of claims individually or in combination. In the figures:

### BRIEF DESCRIPTION OF THE DRAWINGS

FIG. 1 shows a piece of sheet metal from which a lancet body including a lancet tip can be cut by means of a laser;

FIG. 2 shows a semi-finished part that was cut from the piece of metal having a lancet body that is partly cut from the ribbon;

FIG. 3 shows the semi-finished part shown in FIG. 2 and a test strip containing test chemicals attached thereto;

FIG. 4 shows an exemplary embodiment of finished lancets;

FIG. 5 shows an exemplary embodiment of a lancet supply ribbon having multiple lancets according to the exemplary embodiment shown in FIG. 4, with the lancets each being sealed in chambers of a film wrapping;

FIG. 6 shows an exemplary embodiment of a puncturing device with lancet supply ribbon according to FIG. 5;

FIG. 7 shows another exemplary embodiment of a lancet according to the invention residing in a film wrapping;

FIG. 8 shows the exemplary embodiment shown in FIG. 7 after it has been twisted out of the film wrapping, for receiving a sample;

FIG. 9 shows the exemplary embodiment shown in FIGS. 7 and 8 residing in a measuring position after its use;

FIG. 10 shows a section of a metal ribbon with boundary strips defining a region for containing test liquids;

FIG. 11 shows a cross-sectional view related to FIG. 10;

FIG. 12 shows the metal ribbon according to FIG. 10 with applied liquid;

FIG. 13 shows a cross-sectional view related to FIG. 12;

FIG. 14 shows the metal ribbon after the liquid has dried; and

FIG. 15 shows a cross-sectional view related to FIG. 14.

Corresponding reference characters indicate corresponding parts throughout the several views. Although the drawings represent embodiments of the present invention, the drawings are not necessarily to scale and certain features may be exaggerated in order to better illustrate and explain the present invention. The exemplification set out herein illustrates embodiments of the invention, and such exemplifications are not to be construed as limiting the scope of the invention in any manner.

### DETAILED DESCRIPTION OF EMBODIMENTS OF THE INVENTION

For the purposes of promoting an understanding of the principles of the invention, reference will now be made to the embodiments illustrated in the drawings, which are described below. It will nevertheless be understood that no limitation of the scope of the invention is thereby intended. The invention includes any alterations and further modifications in the illustrated devices and described methods and further applications of the principles of the invention, which would normally occur to one skilled in the art to which the invention relates.

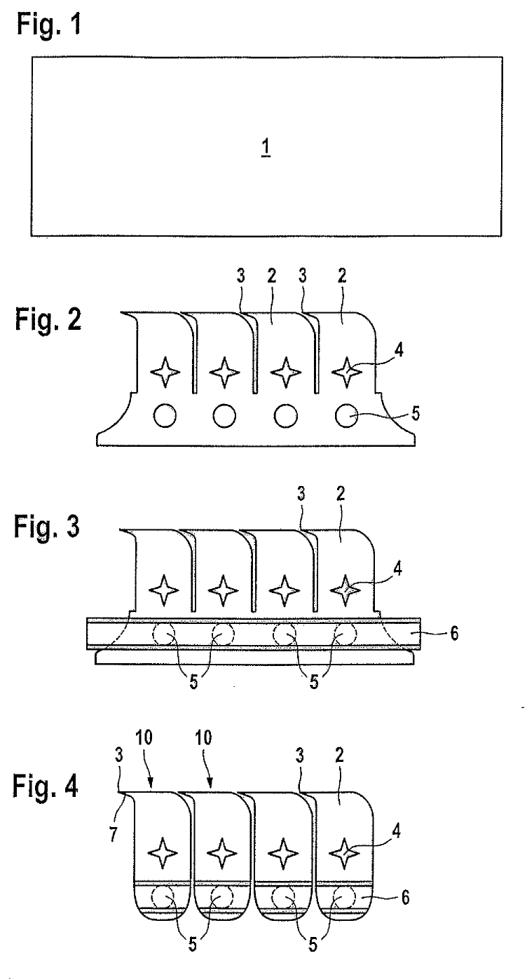

An exemplary embodiment of a method for the manufacture of an exemplary embodiment of a flat lancet shall be illustrated with reference to FIGS. 1 to 4. Lancet bodies 2 including lancet tips 3 may be cut by means of laser cutting from the metal ribbon 1 shown in FIG. 1. In the exemplary embodiments shown, the metal ribbon 1 is a piece of sheet metal with a thickness of about 50 μm or 60 μm to about 100 μm or 200 μm. Any suitable laser, such as Nd:YAG (neodymium-doped yttrium aluminum garnet) laser may be used for laser cutting.

In cutting the metal ribbon 1, it useful to move the metal ribbon 1 with respect to the laser beam although, basically, it is obviously also feasible to move the focus of the laser beam over the surface of the metal ribbon 1. Another method, aside from fusion cutting and sublimation cutting, that may be utilized for cutting the metal ribbon 1 is torch cutting. In torch cutting, oxygen is used as the cutting gas in order for oxidation to increase the erosive effect and thus the effectiveness of a laser beam of given power. The oxygen can be blown through a nozzle to the focus at which the laser beam impacts on the metal ribbon 1. In embodiments of the invention, any suitable process gases or gas mixtures can be used in place of oxygen. It is also feasible to refrain from the use of process or cutting gases.

In a first working step, only a part of the contour of the lancet bodies 2 is cut from the metal ribbon 1 initially such that the semi-finished ribbon shown in FIG. 2 is generated. In the process, two openings 4, 5 are cut into the lancet body 2. The opening (or coupling element) 4 is a hole whose contour deviates from a circular shape. Coupling element 4 is star-shaped in the exemplary embodiment shown. The opening 4 forms a coupling element for coupling to a lancet drive of a puncturing device. A lancet can be made to perform a rotational motion by means of the coupling element 4. This is explained in more detail in the following by means of FIGS. 6 to 9.

The opening 5 of the lancet body 2 shown in FIG. 2 is a circular hole for the test field, but opening 5 may be any suitable shape. In order to provide the test field, a strip 6 containing test chemicals for assaying a body fluid sample is applied to the metal ribbon in a further procedural step, by laminating it on, for example by gluing it on. FIG. 3 shows the semi-finished product shown in FIG. 2 having strip 6 glued onto it. Upon contacting a body fluid sample, the test chemicals effect a change of color of the strip 6 the intensity of which depends on the concentration of the analyte, for example glucose, to be determined. Suitable test chemicals for photometric concentration determinations are used in commercial test elements that are used, for example, for measuring the glucose concentration, and therefore need no further explanation. For assaying a body fluid sample, the sample is applied to the test field 5 such that the strip 6 gets wetted. The strip 6 can be cut to size in a preparatory procedural step using the same laser that is used to cut out the lancet body from the metal ribbon 1.

After affixing the strip 6 containing the test chemicals, the remaining contour of the lancet body 2 is cut out in a further procedural step such that the lancets 10 shown in FIG. 4 result. Generally, the strip 6 containing the test chemicals faces the laser beam during the cutting process such that, upon cutting, first the strip 6 and then the piece of sheet metal 1, being underneath the strip 6 in the direction of the beam, is cut.

The strip 6 containing the test chemicals contains polymer threads that may form a fleece, felt or fabric. At the edges of the strip 6, the polymer threads are fused by melting by means of the laser and pilling is thus prevented.

The lancet tip 3 may be made to be beveled in a further working step. The lancet tip 3 is flattened in the process. By this means, an edge 7 of the lancet tip 3 shown in FIG. 4 can be provided in the form of a blade. For example, the lancet tip 3 and/or one of its edges can be slanted or angled with respect to a plane of the lancet body 2.

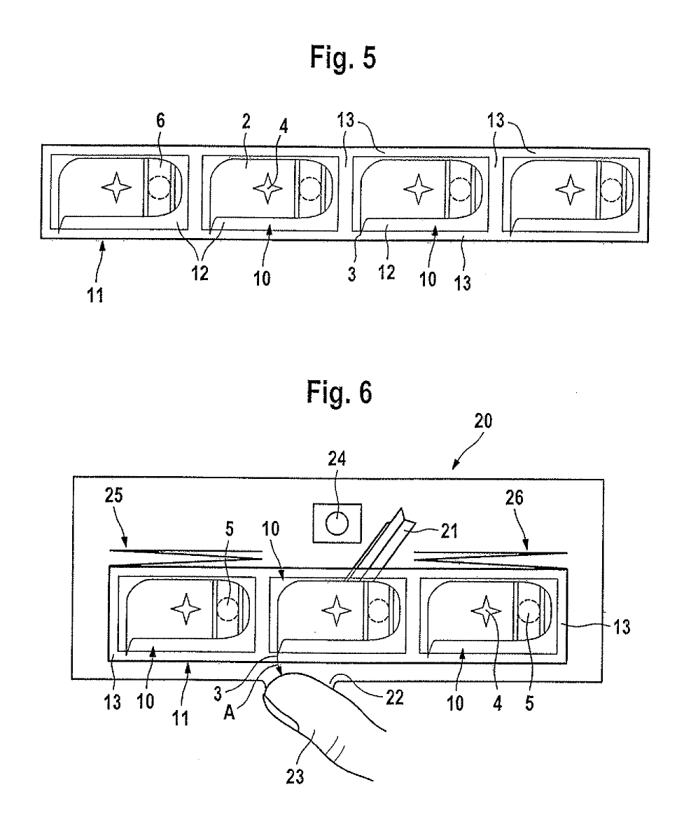

In a further step, the finished lancets 10 are sealed in chambers of a film wrapping 11 that is shown in FIG. 5. FIG. 5 shows a lancet supply ribbon having multiple lancets 10 which each are sealed within chambers 12 of a film wrapping 11, whereby the chambers 12 of the film wrapping form a ribbon. The film wrapping 11 for the lancet supply ribbon shown in FIG. 5 can, for example, be provided in the form of a blister wrapping or two films situated one on top of the other or a film strip that is folded in longitudinal direction can be sealed along the welding seams 13 shown in FIG. 5 in order to effect airtight sealing of the chambers 12 thus formed. Plastic films are particularly well-suited for the film wrapping 11 of the lancets 10, in particular, those made from thermoplastic materials. The welding seams 13 can be generated by means of the same laser that is also used for laser cutting of the metal ribbon 1. In this context, it is useful to move the laser beam by means of a suitable optical set-up, for example a scanner optical set-up, along the welding seams 13 that are to be generated. However, basically, it is also feasible to replace the welding seams 13 shown in FIG. 5 by adhesive connections.

FIG. 6 shows a schematic view of an exemplary embodiment of a puncturing device 20 for generating a puncturing wound, in which a lancet supply ribbon according to FIG. 5 is inserted. The puncturing device 20 for generating a puncturing wound by means of a replaceable lancet 10 shown in FIG. 6 comprises a lancet drive 21 for moving a lancet 10 that is inserted in the device 20 for the purpose of a puncture, whereby the lancet drive 21 transmits a torque to an inserted lancet 10 for the purpose of a puncture such that the lancet 10 performs a puncturing motion in the form of a rotational motion. The rotational motion proceeds about a geometric rotation axis that extends through the coupling element 4 of the lancet 10.

The lancet drive 21 has a drive element in the form of a shaft 21 in the depicted embodiment, whose shape matches a recess forming the coupling element 4, a star-shape in the case of the exemplary embodiment shown, and which is plugged into the coupling element 4 for the purpose of coupling. The drive element 21 is pointed such that it can easily be made to penetrate through the film wrapping 11. By this means, the geometric axis of the rotational motion extends perpendicular to the plane lancet body 2.

A rotational motion of the drive element 21 effects a rotational motion by a few degrees (preferably at least 15°) of the lancet 10 in the direction of the arrow A shown such that the lancet tip 3 generates a puncturing wound in a body part 23 that gets touched to a device opening 22. In the course of a puncturing motion, the lancet tip 3 performs a rotational motion within a plane. The rotational motion of the lancet tip 3 is arc-shaped, in particular circular. For this purpose, it is useful for the coupling element 4 of the lancet 10 to be arranged such that the center of gravity of the lancet 10 resides in the coupling element 4, as is the case in the exemplary embodiment shown. For this reason, the recess of the lancet 10 forming the coupling element 4 is arranged such that it contains the physical center of gravity of the lancet 10 such that unbalance moments are minimized. Performing a rotational motion, the lancet tip 3 cuts through the film wrapping 11 such that the lancet 10 is protected from detrimental environmental influences until its use. Upon this rotational motion for cutting the wrapping 11, the lancet tip 3 first reaches a preparatory position (not shown). Typically, the lancet 10 remains at rest in the preparatory position for several seconds prior to being accelerated for the puncturing motion, i.e. before it is rotated further. By this means, it becomes feasible to prevent the wrapping from affecting the motion speed during the actual puncturing process. However, basically, it is also feasible to open the film wrapping 11 only during the puncturing motion, i.e. to refrain from first rotating the lancet into a preparatory position and penetrating the wrapping in this process.

A puncture is performed by means of a rapid puncturing and returning motion. Accordingly, the direction of rotation of the lancet 10 is reversed by means of the lancet drive 21 as soon as the lancet tip 3 reaches the predetermined depth of puncturing.

After the puncture is made, the lancet tip 3 is rotated away from the device opening 22 and the opposite end of the lancet 10 at which the test field 5 is arranged is rotated out of the device opening 22 such that the lancet becomes situated in a sample reception position and a body fluid sample obtained from the puncturing wound thus generated can be applied to the test field 5 for assaying.

In the exemplary embodiment shown, subsequent to the application of a body fluid sample to the test field 5, the lancet 10 is rotated by the lancet drive 21 by approximately 180° such that the test field 5 reaches a measuring facility 24 that is integrated into the puncturing device 20 and can be used to photometrically detect and analyze a change of color of the test field 5. A test field for electrochemical concentration determination can be used as an alternative to a test field containing test chemicals whose change of color is analyzed by photometry.

It should be noted in the depicted embodiment of the lancet, a body fluid sample may be supplied to the test field 5 only after the lancet tip 3 has been pulled out of a puncturing wound that has been generated. For this reason, the coupling element 4 is arranged between the lancet tip 3 and the test field 5.

In the exemplary embodiment shown, the strip 6 forms a sample reception facility for the reception of a body fluid sample to be assayed. By this means, the sample reception facility is integrated into the test field. The sample reception facility 6 is arranged at a distance from the lancet tip 3 such that a sample reception process can proceed only after the lancet tip 3 has been pulled out of a puncturing wound that has been generated. A sample reception facility of this type can also be provided, for example, in the form of a capillary channel that extends from an edge of the lancet or a sample application field to the test field 5.

In the depicted embodiment of the invention, the sample reception facility 6 is arranged such as to be closer to the coupling element 4 than to the lancet tip 3. In embodiments of the invention, the sample reception facility 6 may be spaced at a distance of about 2 mm or more from the lancet tip 3.

In embodiments of the invention, at least one geometric point of sample reception facility 6 is situated at a distance from the coupling element 4 that corresponds to between about 0.4 and about 0.7 times the distance from the lancet tip 3 and the coupling element 4. Moreover, in embodiments of the invention the at least one geometric point of the sample reception facility 6 is situated at a distance from the lancet tip 3 that is at least as large as the distance between the lancet tip 3 and the coupling element 4. In embodiments of the invention, a geometric point of the area covered by the sample reception facility 6 may be situated at a distance from the coupling element 4 that is approximately equal to the distance between the lancet tip 3 and the coupling element 4. This geometric point may be situated at a distance from the lancet tip 3 that is at least as large as the distance between the lancet tip 3 and the coupling element 4. The distance between the lancet tip 3 and the coupling element 4, as in the exemplary embodiment shown, may correspond to between about 1.5 and about 1.9 times the distance between the coupling element 4 and the test field 5. The lancet tip 3 shall be understood to be the front-most point of the lancet in the direction of puncturing during a puncture.

Measuring facility 24 may be arranged to allow the analysis of the test field 5 to proceed in the sample reception position. In embodiments of the invention, there is no need to have another rotational motion of the lancet, in which the test field 5 containing a sample applied to it must be moved through the film wrapping 11.

In the puncturing device 20 shown, unused lancets 10 of the lancet supply ribbon shown in FIG. 5 are stored in a stack 25 and used lancets are stored in a second stack 26. In the depicted embodiment, the two stacks 25, 26 are arranged in the interior of the device 20 to the left and right of the device opening 22, respectively. In this context, the film wrapping 11 of the lancet supply ribbon shown in FIG. 5 is folded along each of the welding seams 13 that extend transverse to the direction of the ribbon. The puncturing device 20 has a receptacle for insertion of a lancet supply ribbon containing multiple lancets 10, and a transport mechanism for moving an inserted lancet supply ribbon in order to convey lancets 10 of the lancet supply ribbon one after the other into a utilization position in which the coupling element 4 of the lancet 10 is coupled to the lancet drive 21. It is also feasible, for example, to reel the lancet supply ribbon within the device onto a driven roller and provide the transport mechanism accordingly. A roller of this type for reeling used sections of the lancet supply ribbon can be combined with a stack 25 that is formed by unused sections of the lancet supply ribbon, or with a second roller onto which the part of the lancet supply ribbon containing unused lancets is reeled.

It should be noted that a lancet 10 can be rotated back to its original position after its use, in which position it is enveloped by the film wrapping 11 such that used lancets 10 can be stored in the puncturing device 20 in a hygienic fashion and the risk of injuries from lancet tips 3 is reduced during the disposal of a used lancet supply ribbon. In the exemplary embodiment shown, the lancet 10 can be moved by means of a single drive element in order to perforate the wrapping 11, perform the puncturing motion, assume the measuring position, and, after the concentration determination, be safely returned to the wrapping 11 in a hygienic fashion.

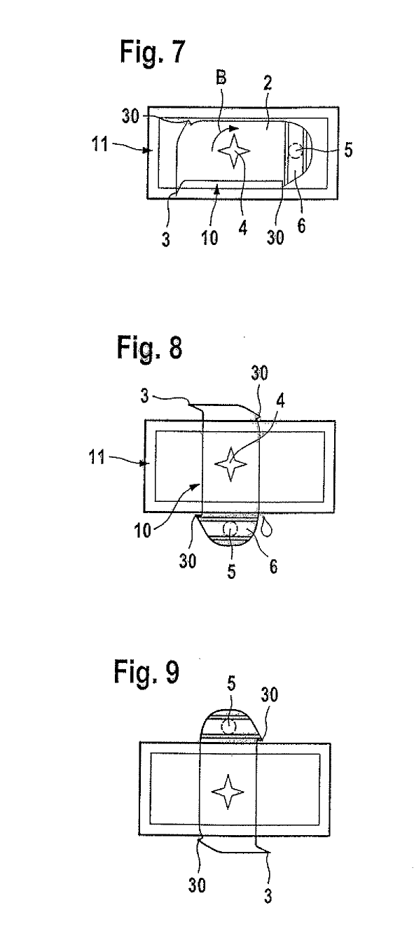

FIG. 7 shows another exemplary embodiment of a lancet 10 in a film wrapping 11. The exemplary embodiment shown in FIG. 7 differs from the preceding exemplary embodiment in that, in addition to the lancet tip 3 for generating a puncturing wound, spikes 30 are arranged on the lancet body 2 for ripping open the film wrapping 11 enveloping the lancet 10. In order to rip open the film wrapping 11, the lancet 10 is first rotated in a first direction of rotation that is shown by an arrow B in FIG. 7. During this first rotational motion, the sides of the film wrapping 11 are slit open by the two spikes 30. Subsequently, the lancet 10 is used to generate a puncturing wound by means of a rotational motion in the opposite direction of rotation. The lancet drive preferably is a spring drive whose spring is tightened upon the first rotational motion in the direction of arrow B.

Slitting open the film wrapping 11 by means of the spikes 30 allows the subsequent puncturing motion of the lancet tip 3 to be performed without any impediments and without the lancet tip 3 having to penetrate through the film wrapping. Moreover, any compromise of the sharpness of the lancet tip 3 that might occur when ripping open the film wrapping 11 may be prevented.

After the puncturing motion has occurred, the lancet 10 is rotated to the position shown in FIG. 8, in which position the test field 5 projects from the device opening 22 such that a body fluid sample can be applied to the test field 5. By means of a subsequent rotation, that corresponds to 180° in the exemplary embodiment of a puncturing device 20 shown in FIG. 6, the test field 5 can be rotated into the interior of the device 20 such that its change of color can be analyzed by means of the measuring facility 24. In FIG. 9, the position of the lancet 10 during the measurement of a change of color of the test field 5 is shown for the exemplary embodiment of a puncturing device 20 shown in FIG. 6. After the measurement was performed, the lancet 10 is rotated back to its original position that is shown in FIG. 7, in which it is stored until disposal. As mentioned above, it is also feasible to perform the measurement in the sample reception position shown in FIG. 8 such that the position shown in FIG. 9 does not need to be assumed in an embodiment that is designed differently form the exemplary embodiments shown in FIG. 6 with respect to the arrangement of the measuring facility 24.

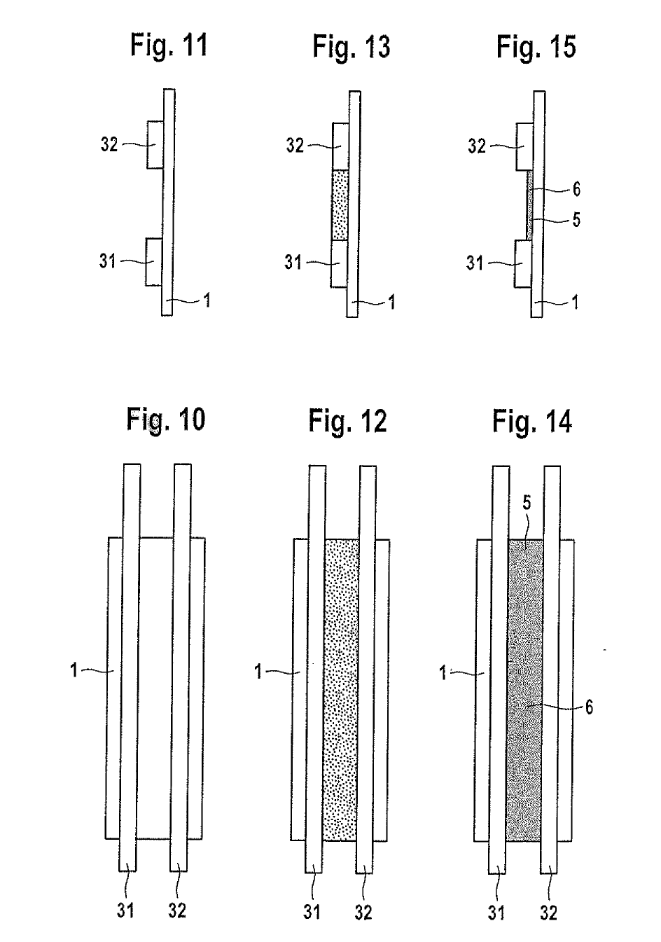

Instead of gluing a strip 6 containing test chemicals to a lancet body 2 or a metal ribbon 1 in order to provide the test field 5, it is also feasible to apply a paste or liquid containing test chemicals to the metal ribbon 1 or the lancet body 2 that is cut from the metal ribbon 1 such that this paste, after drying, forms the test field 5 and a sample reception facility. This procedure is described in the following by means of the FIGS. 10 to 15.

FIG. 10 shows schematically a section of a metal ribbon 1 from which a lancet body 2 and lancet tip 3 is cut by means of laser cutting, as has been illustrated above. In order to provide the test field 5 of the lancets 10 to be cut out, two boundary strips 31, 32 are glued onto the metal ribbon 1 initially. The boundary strips 31, 32 usually are provided in the form of narrow plastic strips that are arranged on the metal ribbon 1 such as to be substantially parallel to each other at a distance of, for example, about 1 mm to about 3 mm. FIG. 11 shows a section of the metal ribbon 1 with boundary strips 31, 32 glued onto it. In a further procedural step, a paste or liquid containing test chemicals for photometric concentration determination is applied between the two boundary strips 31, 32. FIG. 12 shows a top view of a section of the metal ribbon 1 with boundary strips 31, 32 glued onto it and a gap in which the liquid or paste containing the test chemicals is filled. FIG. 13 shows a cross-sectional view related to FIG. 12.

Subsequently, the applied liquid or paste is fully dried such that a strip 6 containing the test field 5 forms between the boundary strips 31, 32. The thickness of the strip 6 that remains between the boundary strips 31, 32 after drying is complete depends on the solids content of the applied paste or liquid. FIG. 15 shows a cross-sectional view of the situation after drying of the paste that was applied between the boundary strips 31, 32. The view presented in FIG. 15 is not to scale. The thickness of the boundary strips 31, 32 is, for example, about 20 μm, and the thickness of the strip 6 containing test chemicals that is formed by drying of the test chemicals is, for example, about 10 μm. The thickness of the metal ribbon 1, for example, is about 80 μm. FIG. 14 shows a top view related to FIG. 15.

A hole 5, such as the one shown in FIG. 2, is not present in lancets having a test field 5 that was manufactured according to the method described by means of FIGS. 10 to 15. For this reason, the measuring facility 24 for photometric analysis of a change of color of the test field 5 resides on the same side of the metal ribbon 1 as the test field 5 during the analysis. However, if a strip 6 according to the methods described by means of FIGS. 1 to 4 is glued over a hole 5 in a lancet body 2, the hole in the lancet body 2 allows the photometric measuring facility to be arranged on the other side such that the lancet body 2 resides between the test strip 6 and the measuring apparatus.

While the invention has been taught with specific reference to these embodiments, one skilled in the art will recognize that changes can be made in form and detail without departing from the spirit and scope of the invention. The described embodiments are to be considered, therefore, in all respects only as illustrative and not restrictive. As such, the scope of the invention is indicated by the following claims rather than by the description.

1. A puncturing device for generating a puncturing wound comprising:
a lancet; and
a drive coupled to the lancet by way of a coupling element;
wherein the lancet rotates about an axis extending through the coupling element in order to generate the puncturing wound.

2. The puncturing device according to claim 1 further including a receptacle configured for the insertion of a lancet supply ribbon including a plurality of lancets and a transport mechanism for moving the inserted lancet supply ribbon in order to convey the plurality of lancets of the lancet supply ribbon into a utilization position in which the coupling element of the lancets is coupled to the lancet drive.

3. A lancet for a puncturing device capable of generating a puncturing wound, the lancet comprising:
a lancet body including a lancet tip and a coupling element configured to couple with the lancet drive in order to cause the lancet to move in a rotational direction; and
a test field for assaying a body fluid sample;
wherein the body fluid sample is obtained after the lancet tip has been removed from the puncturing wound.

4. The lancet according to claim 3 wherein the lancet body carries a sample reception facility for receiving the body fluid sample to be assayed

5. The lancet accordingly to claim 4 wherein the sample reception facility is arranged at a distance from the lancet tip such that sample is received after the lancet tip has been removed from the puncturing wound.

6. The lancet according to claim 4 wherein the sample reception facility is at a distance of at least about 2 mm from the lancet tip.

7. The lancet according to claim 4 wherein the sample reception facility is arranged closer to the coupling element than to the lancet tip.

8. The lancet according to claim 4 wherein the sample reception facility covers a portion of the lancet body, whereby at least one geometric point of the covered portion is situated at a distance from the coupling element that corresponds to between about 0.4 and about 0.7 times the distance between the lancet tip and the coupling element, and whereby this geometric point of the covered point is situated at a distance from the lancet tip that is at least as large as the distance between the lancet tip and the coupling element.

9. The lancet according to claim 3 wherein the coupling element is arranged between the lancet tip and the test field.

10. The lancet according to claim 3 wherein a distance between the lancet tip and the coupling element corresponds to between about 1.5 and about 1.9 times the distance between the coupling element and the test field.

11. The lancet according to claim 3 wherein the coupling element is an opening with a non-circular shape.

12. The lancet according to claim 3 wherein the lancet body further includes at least one spike configured to pierce a wrapping enveloping the lancet.

13. The lancet according to claim 3 wherein the test field is a strip.

14. The lancet according to claim 13 wherein at least a section of the strip contains test chemicals for assaying a body fluid sample.

15. A lancet supply ribbon comprising:
a plurality of lancets comprising a lancet body which has a lancet tip and a coupling element; and
a film wrapping the lancets and forming a ribbon having plurality of chambers containing the lancets.

16. A lancet comprising:
a lancet body;
a lancet tip located proximate a first end of the lancet body;
a test field located proximate a second end of the lancet body; and
a coupling element located intermediate the lancet tip and the test field.

17. The lancet as set forth in claim 16 wherein the coupling element is an opening with a non-circular shape.

18. The lancet as set forth in claim 17 wherein the coupling element has a star shape.

19. The lancet as set forth in claim 16 further including a spike extending outward from the lancet body in a first direction, wherein the lancet tip extends outward from the lancet body in a second direction, and the first direction is substantially opposite from the second direction.

20. The lancet as set forth in claim 16 further including a spike extending outward from the lancet body in a first direction, wherein the lancet tip extends outward from the lancet body in a second direction, and the first direction is substantially the same as the second direction.

21. The lancet as set forth in claim 16 wherein the test field includes a strip attached to the lancet body.

22. The lancet as set forth in claim 21 wherein the strip is laminated onto the lancet body.

23. The lancet as set forth in claim 21 wherein the strip changes color based upon the concentration of glucose in a test sample.

24. The lancet as set forth in claim 21 wherein the strip comprises dried test chemicals applied to the lancet body in a liquid form.

25. The lancet as set forth in claim 24 wherein the test field further includes a pair of boundary members attached to the lancet body, the strip located intermediate the boundary members.

26. The lancet as set forth in claim 21 wherein the test field further includes an opening and the strip covers the opening.

27. The lancet as set forth in claim 16 wherein a first distance separates the lancet tip from the coupling element and a second distance separates the coupling element and a point in the test field, and the second distance is between about 0.4 and 0.7 times the first distance.

28. The lancet as set forth in claim 27 wherein a third distance separates the point in the test field and the lancet tip and the third distance is at least as large as the first distance.

29. The lancet as set forth in claim 16 wherein a first distance separates the lancet tip from the coupling element and the a second distance separates the coupling element from the test field and the first distance is between about 1.5 and 1.9 times greater than the second distance.
